(19) United States
(12) Patent Application Publication
(10) Pub. No.: US 2011/0144587 A1
(43) Pub. Date: Jun. 16, 2011

(54) Liquid agent delivery apparatus, system and method

(76) Inventor: Robert T. Stone, Sunnyvale, CA (US)

(21) Appl. No.: 12/928,349

(22) Filed: Dec. 8, 2010

Related U.S. Application Data
(60) Provisional application No. 61/284,039, filed on Dec. 11, 2009.

(51) Int. Cl. A61M 5/158

(52) U.S. Cl. 604/151

(57) ABSTRACT

A liquid agent delivery apparatus having a hydraulic actuator system, the hydraulic actuator system including a bi-directional pump having a stroke volume less than approximately 100-200 nanoliters, and an agent delivery system that is adapted to cooperate with the hydraulic actuator system, the hydraulic actuator system and cooperating agent delivery system being adapted to jointly deliver approximately 0.05-0.5 micro-liters of a liquid agent out of a skin-piercing agent delivery member per delivery sequence with inlet and outlet pressures at the agent delivery member in the range of approximately −500 to +500 millibars.

### CROSS-REFERENCE TO RELATED APPLICATIONS

This application claims the benefit of U.S. Provisional Application No. 61/284,039, filed Dec. 11, 2009.

### FIELD OF THE PRESENT INVENTION

The present invention relates generally to liquid agent delivery devices. More particularly, the invention relates to pharmacological agent delivery systems and methods for delivering liquid pharmacological or therapeutic agents to a patient.

### BACKGROUND OF THE INVENTION

As is well known in the art, liquid agent delivery devices and systems have numerous uses in the medical field, including subcutaneous delivery of liquid pharmacological and/or therapeutic agents to a patient. For example, a liquid agent delivery device, such as an ambulatory (external) infusion pump is often employed to deliver a defined amount of insulin to a patient diagnosed with diabetes. Such devices are typically worn in a harness or pocket, but can also be strapped to the body of the patient.

Existing liquid agent delivery systems (or pumps) typically include a reservoir, which is adapted to contain the liquid pharmacological agent, and means for achieving fluid-flow communication to a patient or user, e.g., through a suitable hollow tubing. The hollow tubing is typically connected to a hollow needle that is designed to pierce the patient's skin and subcutaneously deliver the liquid agent to the patient. In some delivery systems, the hollow tubing is connected directly to the patient as or through a cannula or set of micro-needles.

The existing liquid agent delivery systems also typically employ electromechanical pumping or metering technology to deliver a defined or prescribed amount of the liquid agent into the hollow tubing and ultimately through the needle or cannula. For example, some liquid agent delivery systems utilize a mechanical peristaltic system whereby mechanical cams are driven by either a rotary motor or an electrical solenoid to compress a flexible tube to deliver a precise dose of a drug. Another liquid agent delivery system mechanically compresses the liquid agent (or drug) reservoir with a screw drive compressor.

Yet another liquid agent delivery system utilizes a shape memory alloy to drive a lead screw and compress a drug storage chamber or reservoir to deliver the liquid agent. The system also incorporates a number of electromechanical actuators and sensors.

The liquid agent delivery systems discussed above are therefore very complex and generally difficult to manufacture. Replacement of the reservoir and skin penetration mechanism is also often difficult. The noted systems, as well as most existing systems, also require specialized care, maintenance, and cleaning to ensure proper functionality and safety.

It is therefore an object of the present invention to provide an improved liquid agent delivery apparatus, system and method that substantially reduces or eliminates the drawbacks and disadvantages associated with convention liquid agent delivery systems.

### SUMMARY OF THE INVENTION

In accordance with the above objects and those that will be mentioned and will become apparent below, the liquid agent delivery apparatus and systems, in accordance with this invention, employ a bi-directional pump with active valves to precisely control the amount and rate of liquid agent delivery without the active pump elements requiring sterilization.

In some embodiments, the liquid agent delivery system (or wearable patch system) comprises a first component comprising a re-usable first housing section having a hydraulic actuator system and a disposable second housing section having a cooperating agent delivery system.

In some embodiments of the invention, the hydraulic actuator system includes a bi-directional pump, a control module (with attendant control/communication electronics) and power supply means.

In some embodiments, the agent delivery system includes a liquid agent containment chamber or reservoir, a plurality of passive check valves (e.g., uni-directional valves), a compressible chamber, skin-piercing means, e.g., a needle, and patient engagement means, e.g., an adhesive area, for securing the liquid delivery system to the patient.

The liquid agent delivery system thus includes a disposable component that can be delivered readily sterilized and a re-usable component, which together provide convenient and precise control of the liquid agent delivery.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a side plane, partial sectional view of a re-usable component (or top section) of a liquid agent delivery system, according to one embodiment of the invention;

FIG. 2 is a bottom plane view of the re-usable liquid agent delivery top component shown in FIG. 1, according to one embodiment of the invention;

FIG. 3 is a schematic illustration of a bi-directional peristaltic pump showing the hydraulic fluid flow paths therein, according to one embodiment of the invention;

FIG. 4 is a top plane view the re-usable liquid agent delivery top component shown in FIG. 1, according to one embodiment of the invention;

FIG. 5 is a side plane, partial sectional view of a disposable component (or bottom section) of a liquid agent delivery system, according to one embodiment of the invention;

FIG. 6 is a top plane view of the disposable liquid agent delivery bottom component shown in FIG. 5, according to one embodiment of the invention;

FIG. 7 is a schematic illustration of a liquid agent delivery system showing the agent flow paths therein, according to one embodiment of the invention;

FIG. 8 is a side plane view of engaged top and bottom components shown in FIGS. 1 and 5, i.e. an assembled liquid agent delivery system, according to one embodiment of the invention;

FIG. 9 is a partial side view of a pump actuation chamber, according to one embodiment of the invention; and

FIG. 10 is an illustration of a human arm having a liquid agent delivery apparatus engaged thereto, according to one embodiment of the invention.

### DETAILED DESCRIPTION OF THE PREFERRED EMBODIMENT

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred apparatus, systems, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a pharmacological agent” includes two or more such agents and the like.

### DEFINITIONS

The term “operational volume”, as used herein, means and includes the volume (or space necessary to contain) the operational, inter-related elements of the hydraulic actuator system and/or agent delivery system, as described herein.

The term “hydraulic fluid”, as used herein, means and includes pure water, silicone oil, or other suitable inert fluid.

The term “liquid agent” as used herein, means and includes a substance, mixture or formulation containing at least one pharmacological agent, as defined herein, which exhibits a characteristic readiness to flow.

The term “pharmacological agent” as used interchangeably herein, and mean and include a biologically active agent, medicament, drug, compound, composition of matter or mixture thereof, including its formulation, which provides some therapeutic, often beneficial effect. This includes any physiologically or pharmacologically active substance that produces a localized or systemic effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

According to the invention, suitable pharmacological agents can comprise or include, for example, inorganic and organic compounds, small molecules, such as steroids and NSAIDs, proteins, enzymes, hormones, oligonucleotides, polynucleotides, nucleoproteins, modified DNA and RNA loaded viruses with modified capsid, polysaccharides, glycoproteins, lipoproteins, polypeptides, including drug carriers, such as pokymers, micro and nano particles.

The pharmacological agent can further comprise or include, without limitation, a biologically active agent selected from the group consisting of leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH (1-24), calcitonin, vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N-[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, VEGF, BNP, ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormones (PTH), parathyroid hormone antagonists, prostaglandin antagonists, pentigetide, protein C, protein S, renin inhibitors, thymosin alpha-1, thrombolytics, TNF, vasopressin antagonists analogs, alpha-1 antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, analgesics, such as fentanyl, remifentanyl, sufentanyl, alfentanyl, lofentanyl, carfentanyl, and analogues and mixtures thereof.

According to the invention, the pharmacological agent can also comprise a vaccine (with or without an antigenic agent), including, without limitation, viruses and bacteria, protein-based vaccines, polysaccharide-based vaccine, and nucleic acid-based vaccines.

Suitable antigenic agents include, without limitation, antigens in the form of proteins, polysaccharide conjugates, oligosaccharides, and lipoproteins. These subunit vaccines include Bordetella pertussis (recombinant PT accince-acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linke to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalovirus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitis, pseudomonas aeruginosa, streptococcus pneumoniae, treponema pallidum, and vibrio cholerae, and mixtures thereof.

Additional commercially available vaccines, which contain antigenic agents, include, without limitation, flu vaccines, lyme disease vaccine, rabies vaccine, measles vaccine, mumps vaccine, chicken pox vaccine, small pox vaccine, hepatitus vaccine, pertussis vaccine, and diptheria vaccine.

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA.

The nucleic acid can also be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct.

The antigens that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include aluminum phosphate gel; aluminum hydroxide; algal glucan: β-glucan; cholera toxin B subunit; CRL1005: ABA block polymer with mean values of x=8 and y=205; gamma inulin: linear (unbranched) β-D (2->1) polyfructofuranoxyl-α-D-glucose; Gerbu adjuvant: N-acetylglucosamine-(β1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c] quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂0 (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; sclavo peptide: VQGEESNDK•HCl (IL-1β 163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

It is also to be understood that more than one agent can be combined or mixed together and incorporated into or used by the present invention, and that the use of the term “pharmacological agent” in no way excludes the use of two or more such “pharmacological agents”.

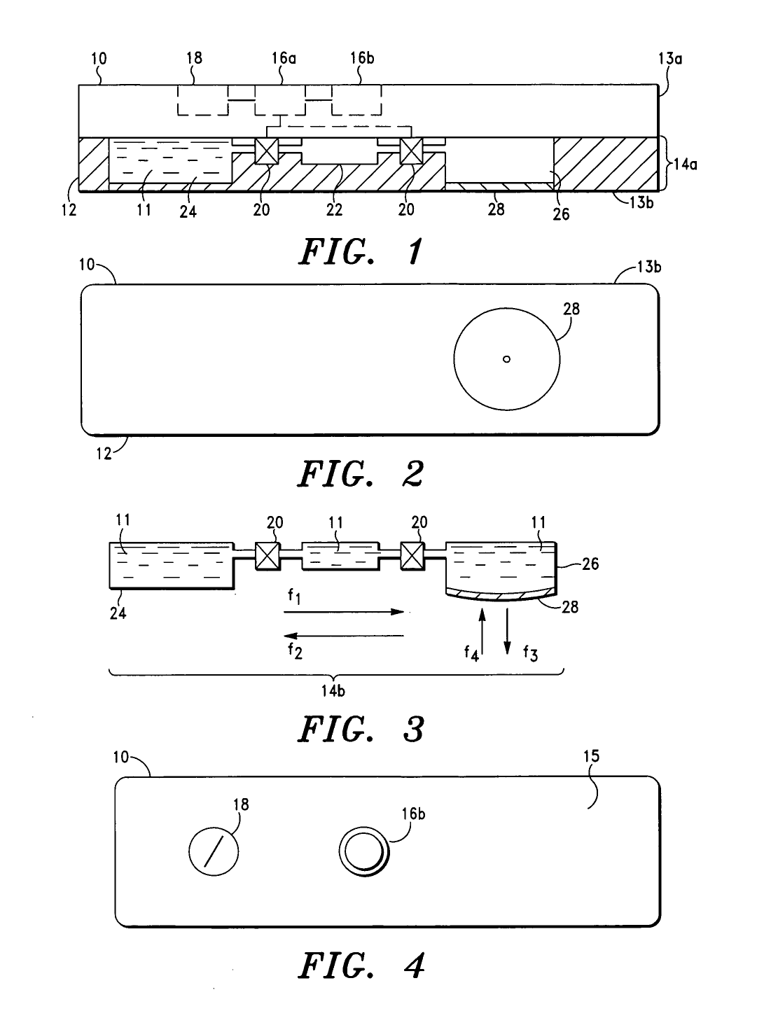

Referring now to FIGS. 1-8, the liquid agent delivery apparatus, systems and methods will be described in detail. As discussed in detail below, in some embodiments of the invention, the liquid agent delivery apparatus and systems of the invention employ a bi-directional peristaltic pump that has a small stroke volume and provides precise liquid agent delivery, bi-directional operation, and minimal, if any, back-leakage.

As will readily be appreciated by one having ordinary skill in the art, the ultra-miniature capabilities of the liquid agent delivery apparatus and systems of the invention provide simple, but highly effective pharmacological agent delivery systems that exceed most performance goals.

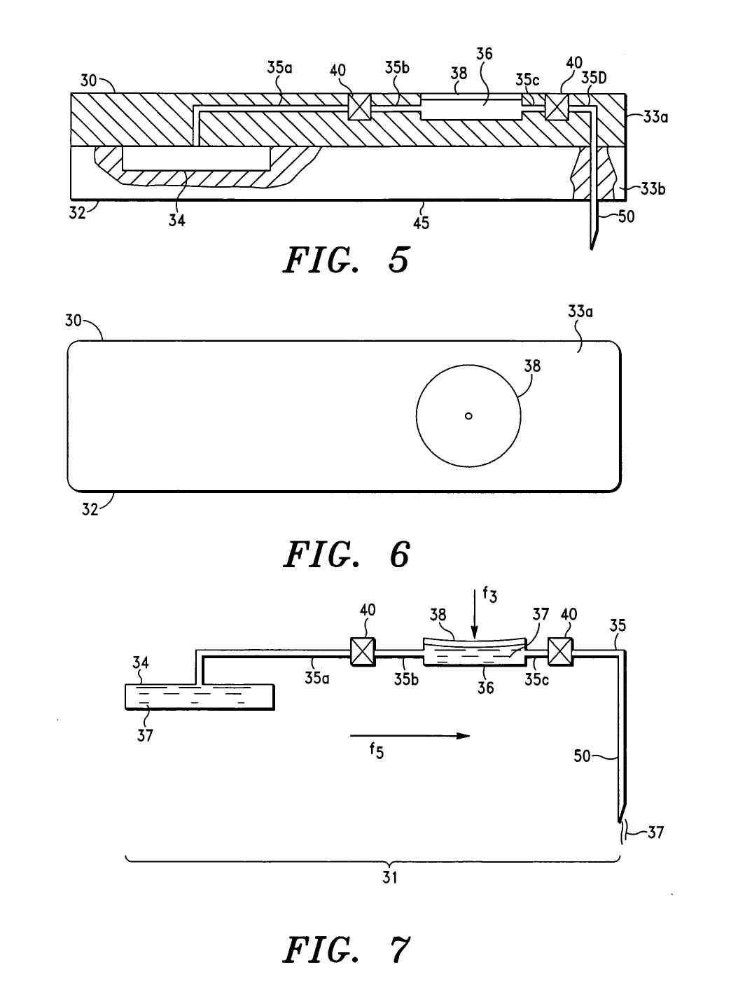

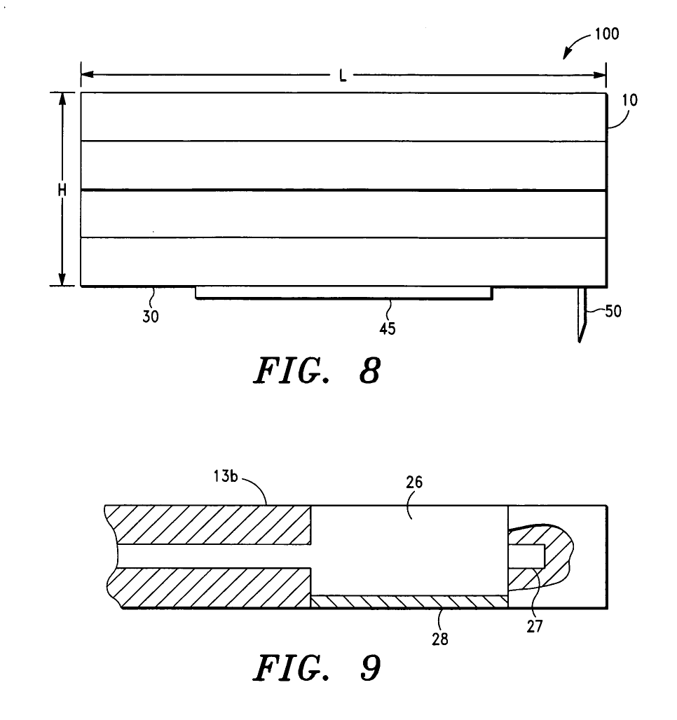

Referring first to FIGS. 1 and 5, there are shown side plane views of the top 10 (FIG. 1) and bottom 30 (FIG. 5) housing sections or components of one embodiment of a liquid agent delivery system 100 of the invention (see FIG. 8). According to the invention, the top and bottom housing components 10, 30 include housings 12, 32, respectively, that are adapted to receive the pump components (discussed herein).

In the illustrated embodiment, the top housing 12 comprises a two-piece unit having a top 13a and bottom 13b section. The bottom housing 32 similarly comprises a two-piece unit having a top 33a and bottom 33b section.

According to the invention, the top and bottom housings 12, 32 can comprise any suitable, preferably, light weight material, such as a high strength polymeric material. In some embodiments of the invention, the housings comprise poly-carbonate, polysulfones, or similar polymers and co-polymers.

According to the invention, the top housing component 10 preferably comprises a re-usable component. By the term “reusable, as used herein, it means that the top housing component 10 can be used multiple times on the same or another patient without the need for sterilization.

In one embodiment of the invention, the top housing component 10 includes a self-contained hydraulic actuator system 14a, having a bi-directional pump (denoted generally “14b” in FIG. 3), control electronics 16a, having function regulating means associated therewith, power supply means 18 (the control electronics 16a and power supply 18 being shown in phantom in FIG. 1), and associated actuator system components, including active stop valves 20 and pump cavity 22.

In a preferred embodiment, the bi-directional pump 14b comprises a piezoelectric, peristaltic pump. In some embodiments of the invention, the peristaltic pump 14b has a stroke volume no greater than 100-200 nanoliters. In some embodiments, the pump 14b has a stroke volume in the range of approximately 50-100 nanoliters.

In some embodiments of the invention, the function regulating means comprises programmed, e.g., automated, regulating means. In some embodiments, as shown in FIG. 4, the function regulating means comprises (or includes) manual regulating means 16b, e.g., an on-off switch.

In some embodiments, the power supply means 18 comprises a 4.0 volt lithium battery.

As illustrated in FIG. 4, in some embodiments of the invention, the function regulating means 16b and power supply means 18 are disposed proximate the top surface 15 of the top housing component 10.

In some embodiments of the invention, the control electronics 16a are adapted to at least monitor and regulate the bi-directional operation of the pump 14b, e.g., hydraulic fluid threshold (discussed herein), and receive and/or acquire and/or transmit data concerning dosage administered, patient compliance, and device performance parameters. In a preferred embodiment, the control electronics 16a are also in communication with and adapted to regulate stop valves 20 and check valves 40 (shown in FIGS. 3 and 7, and discussed below).

In some embodiments of the invention, the hydraulic actuator system 14a and cooperating agent delivery system 31 (discussed below) are adapted to deliver precise amounts of liquid agent into, through and out of the agent delivery member 50 (see FIG. 5) in the range of approximately 0.05-0.5 micro-liters per increment, i.e. liquid agent delivery sequence, with inlet and outlet pressures at the agent delivery member 50 in the range of approximately −200 to +200 millibars. In some embodiments, the hydraulic actuator system 14a and cooperating agent delivery system 31 are adapted to deliver precise amounts of liquid agent into, through and out of the agent delivery member 50 in the range of approximately 0.05-0.5 micro-liters per increment with inlet and outlet pressures at the agent delivery member 50 in the range of approximately −200 to +200 millibars.

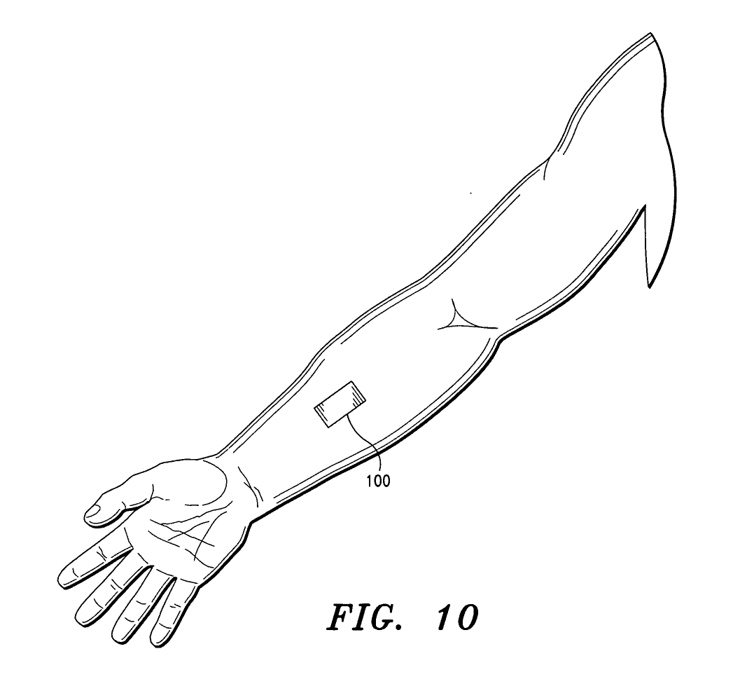

As illustrated in FIG. 1, the hydraulic actuator system 31 (or top housing component 10) also includes a hydraulic fluid reservoir 24 that is adapted to contain a hydraulic fluid 11 therein and a hydraulic actuator chamber 26 having a flexible membrane 28 associated therewith (see also FIG. 9).

Referring now to FIG. 3, according to one embodiment of the invention, during operation, the hydraulic fluid 11 is actively pumped in precisely timed amounts and volumes from the reservoir 24 to the actuator chamber (denoted by Arrow f₁) 26 via the peristaltic pump 14.

In a preferred embodiment, when the volume of the hydraulic fluid 11 that is pumped to the actuator chamber 26 exceeds a predetermined threshold, the bi-directional pump 14b is reversed and returns the hydraulic fluid 11 from the actuator chamber 26 to the hydraulic fluid reservoir 24 (denoted by Arrow f₂).

In some embodiments of the invention, the hydraulic fluid threshold is in the range of 500-1000 micro-liters. In some embodiments of the invention, the hydraulic fluid threshold is in the range of 100-200 microliters.

Referring now to FIGS. 5 and 7, the bottom housing section 30 preferably comprises a disposable component having an agent delivery system 31 comprising a liquid agent reservoir 34, a liquid agent pumping chamber 36, having a flexible membrane 38 associated therewith, check valves 40, interconnecting agent delivery pathways 35a-35d and a fluid delivery member (or skin-piercing injection member or cannula) 50.

In a preferred embodiment of the invention, the liquid agent reservoir 34 is adapted to contain at least 2.5 cc of a liquid agent 37 therein. In some embodiments, the liquid agent reservoir 34 is adapted to contain in the range of approximately 3.0-5.0 cc of liquid agent 37 therein.

According to the invention, the flexible pumping chamber membrane 38, as well as the actuator membrane 28, discussed above, can comprise any suitable flexible material, including silicone, poly-urethane, and PPSU. In one embodiment, the membranes 38, 28 comprise silicon.

According to the invention, the fluid delivery member 50 can be constructed out of any suitable material, such as stainless steel, titanium, nickel titanium alloys, or similar biocompatible materials, such as polymeric materials.

In a preferred embodiment of the invention, the bottom housing component 30 further includes patient engagement means 45, e.g., an adhesive area, for securing the fluid delivery system to a patient.

Referring now to FIG. 8, in a preferred embodiment, the bottom housing component 30 is adapted to receive and seat the top housing component 10. According to the invention, various conventional mechanical and chemical means, e.g., adhesives, can be employed the engage the top 10 and bottom 30 housing components.

In a preferred embodiment, the liquid agent delivery apparatus and systems of the invention have an operational volume, i.e. the space necessary to contain the operational, inter-related elements of the hydraulic actuator system 14a and agent delivery system 31, less than approximately 25 cc.

In some embodiments of the invention, the top 10 and bottom 30 housing components have a joined or engaged (see FIG. 8) total housing volume (i.e. size) less than approximately 40 cc.

In some embodiments of the invention, the top 10 and bottom 30 housing components have a joined or engaged length no greater than 75 mm, width no greater than 50 mm, and height no greater than 10 mm.

According to the invention, when assembled, i.e. top 10 and bottom 30 housing components operatively engaged, the top (or re-usable) housing component 10 is preferably in close physical contact with the bottom (or disposable) housing component 30. In a preferred embodiment, the hydraulic actuator chamber 26, more preferably, the actuator chamber flexible membrane 28, is disposed proximate (more preferably, in contact) with the flexible pumping chamber membrane 38 in the disposable bottom housing component 30.

Referring now to FIGS. 3 and 7, according to one embodiment of the invention, when the volume of the hydraulic fluid 11 in the actuator chamber 26 increases via actuation of the pump 14, and an equal volume of liquid agent 37 is displaced from the liquid agent pumping chamber 36 by the resultant downward deflection of the actuator chamber membrane 28 and, hence, pumping chamber membrane 38 (denoted by Arrow f₃), the liquid agent 37 is delivered to the patient (via fluid delivery member 50) by virtue of check valves 40, which preferably allow only one-way flow through the liquid delivery system 31.

In accordance with one embodiment of the invention, when the hydraulic fluid 11 is pumped from the actuator chamber 26 to the reservoir 24, the resilient actuator chamber membrane 28 (and, hence, pumping chamber membrane 38) lift up, i.e. move in the direction denoted by Arrow f₄, creating suction that allows the liquid agent 37 to be drawn from the liquid agent reservoir 34 and directed to the liquid agent pumping chamber 36 (preferably, through a check valve 40).

When the quantity of liquid agent 37 that is stored in the liquid agent reservoir 34 has been depleted, the disposable bottom housing component 30 is replaced and the re-usable top housing component 10 is re-attached to a new disposable component 30 for further use with the same (see FIG. 10) or another patient.

According to the invention, a number of safeguards are also provided, including a pressure sensor 27 in the hydraulic actuator chamber 26 (see FIG. 9) that can sense either blockage of the patient injection flow path (denoted by Arrow f₅ FIG. 7) or failure of a hydraulic component, e.g., stop valves 20, in the re-usable top housing component 10.

In some embodiments of the invention, the control electronics 16a includes means for monitoring and/or regulating the number of agent delivery sequences and/or number of times the re-usable housing component 10 is re-connected to a disposable housing component 30.

In some embodiments of the invention, the control electronics 16a also include means of ceasing operation of the hydraulic actuator system 14a when a predetermined number of agent delivery sequences and/or number of times the re-usable housing component 10 is re-connected to a disposable housing component 30 is achieved.

Further, the passive check valves 40 can be replaced with active stop valves that are implemented in a similar fashion (as described herein) for the pump 14 or directly with either piezo-electric actuator elements or with electrical solenoid elements. As will be readily apparent to one having ordinary skill in the art, lower operating power can be achieved with the use of passive valves, which enables the development of smaller liquid delivery systems.

In some envisioned embodiments of the invention, the liquid agent delivery apparatus and systems of the invention include wireless signal transmission means for receiving, for example, system control signals, and transmitting system operation signals, e.g., times of application, agent delivery sequences, etc. As will readily be appreciated by one having ordinary skill in the art, the transmitted signals can be transmitted to and stored by various devices for real-time or subsequent assessment by a physician or skilled technician.

As will be readily appreciated by one having ordinary skill in the art, the liquid delivery apparatus and systems of the invention provide numerous advantages. Among the advantages are the following:
The provision of a liquid agent delivery apparatus having a disposable component containing an agent delivery system and a re-usable component having an actuator system (or pump) that can be used multiple times without the necessity of sterilization;
The provision of a liquid agent delivery apparatus having a bi-directional peristaltic pump that provides precise liquid agent delivery, bi-directional operation, minimal, if any, back-leakage, and exhibits a stroke volume less than approximately 100-200 nanoliters;
The provision of a liquid agent delivery apparatus that includes a hydraulic actuator system and cooperating agent delivery system that jointly are capable of delivering precise amounts of liquid agent into, through and out of a skin-piercing agent delivery member in the range of approximately 0.05-0.5 micro-liters per increment with inlet and outlet pressures at the agent delivery member in the range of approximately −500 to +500 millibars;
The provision of a liquid agent delivery apparatus having a total housing volume (i.e. size) less than 40 cc;
The provision of a liquid agent delivery apparatus having an operational volume less than 25 cc;
The provision of a liquid agent delivery apparatus that is adapted to administer a precise amount of a liquid pharmacological agent in the range of 0.5 ul/hr to 20 ul/hr; and
The provision of a liquid agent delivery apparatus that is adapted to administer a precise amount of a liquid pharmacological agent in the range of 1.0 ul to 240 ul in 10 minutes or less.

Potential applications of the liquid delivery apparatus and systems of the invention include, without limitation, the following:
Precise administration of insulin as treatment for diabetes;
Precise administration of baclofenas as treatment for muscle tension;
Precise administration of opiates as treatment for chronic pain;
Precise administration of antibiotics as treatment for local infections; and
Precise administration of cancer therapy drugs as treatment for organ localized cancers.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A liquid agent delivery apparatus, comprising:
a first housing section having a hydraulic actuator system, said hydraulic actuator system including a bi-directional pump having a stroke volume less than approximately 100-200 nanoliters; and
a second housing section having an agent delivery system that is adapted to cooperate with said hydraulic actuator system, said hydraulic actuator system and cooperating agent delivery system being adapted to jointly deliver a liquid agent into, through and out of a skin-piercing agent delivery member in the range of approximately 0.05-0.5 micro-liters per delivery sequence with inlet and outlet pressures at said agent delivery member in the range of approximately −500 to +500 millibars.

2. The agent delivery apparatus of claim 1, wherein said first housing section comprises a re-usable housing component.

3. The agent delivery apparatus of claim 1, wherein said hydraulic actuator system and agent delivery system jointly have an operational volume less than approximately 25 cc.

4. An agent delivery system, comprising:
a re-usable housing section having a hydraulic actuator system and control module,
said hydraulic actuator system including a bi-directional pump, hydraulic fluid distribution line, hydraulic fluid reservoir that is adapted to contain a first volume of hydraulic fluid therein, and pump actuator chamber that is adapted to receive a said hydraulic fluid therein, said hydraulic fluid reservoir and said pump actuator chamber being in communication with said hydraulic fluid distribution line, said pump actuator chamber having a pump actuator chamber membrane that is disposed proximate a first surface of said re-usable housing section and positioned and adapted to transition from a first pump actuator chamber membrane position to a second pump actuator chamber membrane position when said pump actuator chamber receives a second volume of said hydraulic fluid therein, said pump further including at least one stop valve that is in communication with said hydraulic fluid distribution line and is adapted to control hydraulic fluid flow through said fluid distribution line, said stop valve being disposed between said hydraulic fluid reservoir and said pump actuator chamber,
said control module including control means adapted to monitor and regulate bi-directional operation of said bi-directional pump, said control means being further adapted to regulate said pump stop valve, power supply means for providing power to the fluid delivery system, and manual pump regulating means; and
a disposable housing section that is adapted to operationally engage said re-usable housing section, said disposable housing section including an agent delivery system,
said agent delivery system including an agent delivery line, an agent reservoir that is adapted to contain an agent therein, an agent pumping chamber that is adapted to receive said agent therein, and a fluid delivery member, said agent reservoir, said agent pumping chamber and said agent delivery member being in communication with said agent delivery line, said agent pumping chamber including an agent pumping chamber membrane that is disposed proximate a first surface of said disposable housing section whereby when said re-usable housing section is operatively engaged to said disposable housing section said agent pumping chamber membrane is disposed proximate said pump actuator chamber membrane, and whereby when said pump actuator chamber membrane transitions from a first pump actuator chamber membrane position to a second pump actuator chamber membrane position said agent pumping chamber membrane transitions from a first agent pumping chamber membrane position to a second agent pumping chamber membrane position, whereby said agent flows into, through and out of said fluid delivery member,
said agent delivery system further including at least check valve that is in communication with said agent delivery line and adapted to control agent flow through said agent delivery line, said check valve being disposed between said agent reservoir and said agent pumping chamber, said check valve being in communication with and regulated by said pump control means.

5. The agent delivery system of claim 4, wherein when said second volume of said hydraulic fluid received by said pump actuator chamber exceeds a hydraulic fluid threshold in the range of 500-1000 microliters, said pump reverses whereby said hydraulic fluid flows from said pump actuator chamber to said hydraulic fluid reservoir.

6. The agent delivery system of claim 4, wherein when said second volume of said hydraulic fluid received by said pump actuator chamber exceeds a hydraulic fluid threshold in the range of 100-200 microliters, said pump reverses whereby said hydraulic fluid flows from said pump actuator chamber to said hydraulic fluid reservoir.

7. The agent delivery system of claim 5, wherein when said pump reverses, said agent is drawn from said agent reservoir and flows into and through said agent delivery line to said agent pumping chamber.

8. The agent delivery system of claim 4, wherein said pump has a stroke volume less than approximately 100-200 nanoliters.

9. The agent delivery system of claim 4, wherein said hydraulic actuator system and agent delivery system are adapted to jointly delivery in the range of approximately 0.05-0.5 micro-liters per increment of said liquid agent out of said fluid delivery member with inlet and outlet pressures at said delivery member in the range of approximately −500 to +500 millibars.

10. The agent delivery system of claim 4, wherein said hydraulic actuator system and agent delivery system are adapted to jointly delivery in the range of approximately 0.05-0.5 micro-liters per increment of said liquid agent out of said fluid delivery member with inlet and outlet pressures at said delivery member in the range of approximately −200 to +200 millibars.
